(19) United States
(12) Patent Application Publication
(10) Pub. No.: US 2012/0129801 A1
(43) Pub. Date: May 24, 2012

(54) BLOOD PARASITICIDE

(75) Inventors: Rogerio Grassetto Artacho, Sao Paulo (BR); Luis Fernando Vettorato, Sao Paulo (BR); Renato Antonio Beneduzzi Ferreira, São Paulo (BR); João Marcelo de Castro Gomes, São Paulo (BR)

(73) Assignee: Wyeth LLC, Madison, NJ (US)

(21) Appl. No.: 13/318,210

(22) PCT Filed: Apr. 29, 2010
(86) PCT No.: PCT/US2010/032923

§ 371 Date: Jan. 23, 2012

Related U.S. Application Data
(60) Provisional application No. 61/174,811, filed on May 1, 2009.

(51) Int. Cl. A61K 31/714; A61P 33/00

(52) U.S. Cl. 514/52

(57) ABSTRACT

This application relates to compositions and methods of treating tick-born parasitic diseases in livestock with the compositions. The compositions include imidocarb dipropionate, cyanocobalamin vitamin B12, and pharmaceutically acceptable carriers.

### BACKGROUND

1. Field of the Invention

This application relates to a novel formulation for use in the treatment of tick-born parasitic diseases in livestock.

2. Prior Art

Babesiosis is an intraerythrocytic parasitic infection caused by protozoa of the genus Babesia, such as B. bovis and B. bigemina, that is generally transmitted through a tick bite, similar to the mechanism by which Lyme disease is transmitted. See, e.g. BOCK, R., JACKSON, L., DE VOS, A. & JORGENSEN, W. (2004). “Babesiosis of cattle.” Parasitology: 129, Suppl, S247-S269. Anaplasmosis, also a vector-borne, infectious blood disease, is caused by rickesttsial parasites Anaplasma marginale and Anaplasma centrale. See, e.g., KOCAN, K. M., DE LA FUENTE, J., BLOUIN, E. F. & GARCIA-GARCIA, J. C. (2004). “Anaplasma marginale (Rickettsiales: Anaplasmataceae): recent advances in defining host-pathogen adaptations of a tick-borne rickettsia.” Parasitology: 129, S285-S300. These, and other similar parasites, are extremely undesirable in populations of livestock. They are both endemic problems that can flare up to epidemic proportions at any time.

Imidocarb dipropionate is a carbanilide derivative that has been used for many years for the treatment of protozoal diseases, including babesiosis and anaplasmosis, in cattle, horses, sheep, and dogs. See, e.g., MCHARDY, N. & SIMPSON, R. M. (1973). “Imidocarb dipropionate therapy in Kenyan anaplasmosis and babesiosis.” Tropical Animal Health and Production: 6, No. 2, 63-70. See also, e.g. VIAL, H. J. & GORENFLOT, A. (2006). “Chemotherapy against babesiosis.”Veterinary Parasitology: 138, 147-160. The chemical name of imidocarb dipropionate is N,N′-bis (3-(4,5-Dihydro-1H-imidazol-2-yl)phenyl) urea and its structure is as shown in Figure I. It can be used in the form of its dipropionate or hydrochloride salt and can be administered by way of intramuscular or subcutaneous injection to lessen irritability. Imidocarb can also prevent and treat eperythrozoonosis, toxoplasmosis, giardiasis, trypanosomiasis, as well as Theileria annulata in many sorts of animals. It is quickly absorbed and distributed throughout the body after injection. The drug is metabolized in the liver and reabsorbed in original form by the kidney, so its dosage is small and its effect is permanent.

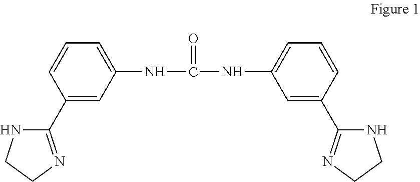

It is understood that imidocarb dipropionate acts by interfering with polyamine synthesis and function within invading parasites. Common adverse side effects associated with known formulations are significant pain upon injection as well as acute toxicity symptoms consistent with a cholinesterase inhibitor activity, excessive salivation, lacrimation, increased frequency of defecation, tachypnea and abdominal pain, sometimes leading to leading to colic.

Vitamin B12 (cyanocobalamin) is involved in protein, carbohydrate and fat metabolism, as well as working in conjunction with folic acid to maintain red blood cell production. Vitamin B12 contains the essential mineral cobalt. It is a known dietary supplement for livestock that is necessary for general metabolism, nervous and reproductive function. It also facilitates blood cells formation and improves hair coat condition. Vitamin B12 injections are an effective and tested cure for patients suffering from a deficiency of the vitamin. The utility of vitamin B12 is not restricted to curing deficiencies. Injections thereof can also serve therapeutic purposes. A vitamin B12 injection acts as a stimulant for energizing the body, through cobalamin, which transmits its “anti-stress” elements.

A known issue with vitamin B12 is its stability in formulations. Vitamin B12 is generally known to be stable to heat but is sensitive to light, oxygen, acid and alkali. Vitamin B12 is also not stable when in an environment containing pro-oxidants. Some ingredients, especially those found in multi-vitamins, will have cross-interactions with the vitamin B12 and destroy it. Further complicating the issue of creating formulations including vitamin B12 is that it is necessary and desirable to use only small quantities of vitamin B12 to have optimal effect.

### SUMMARY OF INVENTION

Disclosed herein are novel formulations comprising imidocarb dipropionate, vitamin B12 and pharmaceutically acceptable carriers that improve the treatment and prevention of animals infected with certain parasitic infections. The novel formulations disclosed and claimed herein offer numerous advantages over the prior art with respect to efficacy, safety, stability and reduction of side effects associated with prior art imidocarb-based therapies. The novel combinations disclosed herein are surprisingly stable despite known difficulties with liquid formulations containing Cyanocobalamin vitamin B12.

The novel formulations disclosed herein combining imidocarb and vitamin B12 provide not only a unique combination, but also added convenience. Treatment in a single injection combining imidocarb and vitamin B12 requires less effort, less materials and less handling of the animal than a treatment wherein the ingredients are administered separately.

The novel formulations disclosed herein are useful both as a treatment for animals already suffering from parasitic infections as well as for prevention. In cases where animals are being introduced to an environment where a tick population is thought to exist that could carry parasites that cause babesiosis, anaplasmosis or other parasitic infections that respond to imidocarb treatment, the formulations disclosed herein are useful to effectively prevent subsequent infection by these parasites.

### DETAILED DESCRIPTION OF THE INVENTION

Disclosed herein are novel formulations including imidocarb dipropionate, vitamin B12 and one or more pharmaceutically acceptable carriers which exhibit improved effects in the treatment and prevention of parasitic infections of livestock.

The formulations of the invention may be administered by any conventional method including parenteral (e.g. subcutaneous or intramuscular) injection or intravenous infusion routes. The treatment may consist of a single dose or a plurality of doses. While it is possible for the imidocarb and vitamin B12 to be administered separately, for obvious reasons it is preferable to present it as a single pharmaceutical formulation, together with one or more acceptable carriers. The carrier(s) must be “acceptable” in the sense of being compatible with the formulation and not deleterious to the recipients thereof. Typically, the carriers will be water or saline which will be sterile. Formulations of the invention may include aqueous carriers such as sterile pyrogen-free water, saline or other isotonic solutions because of their extended shelf-life in solution. Pharmaceutical compositions of the invention may be formulated well in advance in aqueous form, for instance, weeks or months or longer time periods before being dispensed.

Formulations suitable for parenteral administration include aqueous and non-aqueous sterile injection solutions which may contain anti-oxidants, buffers, bacteriostats and solutes which render the formulation appropriate for the intended recipient; and aqueous and non-aqueous sterile suspensions which may include suspending agents and thickening agents. The formulations may be presented in unit-dose or multi-dose containers, for example sealed ampules, vials or syringes, and may be stored in a freeze-dried (lyophilised) condition requiring only the addition of the sterile liquid carrier, for example water for injections, immediately prior to use. Extemporaneous injection solutions and suspensions may be prepared from sterile powders.

Formulations or compositions of the invention may be packaged together with, or included in a kit with, instructions or a package insert referring to the extended shelf-life of the formulation. For instance, such instructions or package inserts may address recommended storage conditions, such as time, temperature and light, taking into account the extended or prolonged shelf-life of the formulations of the invention. Such instructions or package inserts may also address the particular advantages of the formulations of the inventions, such as the ease of storage for formulations that may require use in the field, outside of controlled clinic or office conditions. As described above, formulations of the invention may be in aqueous form and may be stored under less than ideal circumstances without significant loss of therapeutic activity.

The invention also provides methods of treatment and/or prevention of diseases or disorders (such as, for example, any one or more of the diseases or disorders disclosed herein) by administration to a subject of an effective amount of a formulation of the invention in a pharmaceutically acceptable carrier.

The parasiticide formulations will be formulated and dosed in a fashion consistent with good veterinary practice, taking into account the condition of the individual animal, the site of delivery, the method of administration, the scheduling of administration, and other factors known to practitioners. The “effective amount” for purposes herein is thus determined by such considerations.

As a general proposition, the pharmaceutically effective amount of imidocarb the formulations herein administered parenterally will be in the range of about 0.5 mg/kg to 10 mg/kg of animal body weight, although, as noted above, this will be subject to therapeutic discretion. More preferably, this dosage is 3 mg/kg to 6 mg/kg. The length of treatment needed to observe changes and the interval following treatment for responses to occur varies depending on the desired effect. The pharmaceutically effective amount of Cyanocobalamin Vitamin B12 the formulations herein administered parenterally will be in the range of about 3 μg/kg to 100 μg/kg of patient body weight, although, as noted above, this will be subject to therapeutic discretion. More preferably, this dosage is 5 μg/kg to 20 μg/kg.

For example, PEG 400, utilized in the examples as a carrier in the examples herein, is harmless towards skin and easily soluble in water. This makes it an attractive ingredient in an injectible formulation. Those of skill in the art will understand that other such excipients with similar properties could be substituted to create a suitable composition consistent with the claims of the invention. For example, other suspending agents may be other hydrophilic polymers, particularly other polyethylene oxide polymers. As used herein, polyethylene oxide polymers of PEG refers to a polymer having the general formula H(OCH₂CH₂)ₙOH. Generally, each PEG is designated by the average number of “n” units or its average molecular weight in daltons. Various molecular weights of polyethylene glycol polymers are known in the art, including, PEG 200 (n=4), PEG 300 (n=6), PEG 400 (n=8), PEG 600 (n=12), PEG 900, PEG 1000 (n=20), PEG 1450 (n=32), PEG 3350 (n=75), PEG 4500 (n=100), and PEG 8000 (n=150). Preferably, the polyethylene glycol polymers are polymers PEG 200 to PEG 600, which have a range of molecular weights of from about 190 to about 630 daltons. Preferred polyethylene glycols include, but are not limited to, PEG 200, PEG 300, PEG 400, and PEG 600. Other carriers/surfactants that could be useful in the disclosed compositions are propylene glycol fatty acid esters such as, among others, propylene glycol monocaprylate, propylene glycol dicaprylate, propylene glycol dicaprate, propylene glycol dicaprylate dicaprate, propylene glycol dilaurate, propylene glycol hydroxystearate, propylene glycol isostearate, propylene glycol laurate, propylene glycol ricinoleate, propylene glycol stearate, propylene glycol dioctanoate, and propylene glycol ricinoleate. Preferred propylene glycol fatty acid esters include, but are not limited to, C8/C10 triglyceride and propylene glycol dicaprylate dicaprate, and compatible mixtures thereof.

Another excipient that can be used in the disclosed formulations is propionic acid. Propionic acid inhibits the growth of mold and some bacteria at the levels between 0.1 and 1% by weight. Propionic acid is also an organic acid that can create salt forms of the active ingredients. The pharmaceutically acceptable salts can be obtained by including an inorganic acid or an organic acid in suitable solvent. Examples of the inorganic acid include hydrochloric acid, sulfuric acid, nitric acid, phosphoric acid, periodic acid and the like. Further, examples of the organic acid include formic acid, acetic acid, butyric acid, oxalic acid, malonic acid, valeric acid, succinic acid, fumaric acid, maleic acid, tartaric acid, citric acid, malic acid, benzoic acid, p-toluenesulfonic acid, methanesulfonic acid and the like.

Further, as another example, the formulation of Example 2 includes Lidocaine as an ingredient that acts as an anesthetic at the inoculation cite to minimize the discomfort to the animal receiving the injection. Other anesthetics are known in the art and would be suitable as a substitute for or in addition to, lidocaine in the disclosed formulations.

The formulations of this invention are particularly useful in that they are safe, efficacious and exhibit little or no injection site reactions which are both painful to the animal and which potentially damage the meat of a livestock animal intended for use as foods. Further, they exhibit the necessary stability so that they can be formulated, packaged, shipped and stored for a sufficient length of time without a diminution in the amount of actives present in the formulation. Thus provided is a product with a commercially useful shelf life that avoids premixing of the two actives or separate administration of the actives.

The foregoing description of the invention has been presented for purposes of illustration and description. It is not intended to be exhaustive or to limit the invention to the precise form disclosed. Obvious modifications or variations are possible in light of the above teachings. The embodiments were chosen and described to provide the best illustrations of the principles of the invention and the practical applications thereof so as to enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

### Example 1

The following is an example of a formulation consistent with the inventions disclosed herein. Of note, the formulation of Example 1 can be made in large batches.

| INGREDIENT | QUANITY/L | | BATCH QUANTITY | |
|---|---|---|---|---|
| Imidocarb dipropionate | 0.1650 | kg | 8.2500 | kg |
| Polyethylene glycol 400 | 0.0500 | kg | 2.5000 | kg |
| Propionic acid | 0.0400 | kg | 2.0000 | kg |
| Cyanocobalamin Vit-B12 | 0.5500 | g | 27.5000 | g |
| EDTA | 0.1000 | g | 5.0000 | g |
| Sodium chloride P.A | 0.0080 | g | 0.4000 | kg |
| Water for injectables (WFI) | 1.0000 | L | 50.0000 | L |

The ingredients were formulated as follows:

1. Check separately the components of the formula and equipment.

### MIXTURE A

2. Collect 50% of the water volume (WFI) 25 L at a temperature of 25-35° C. Maintain the mixture under nitrogen bubbling.

3. Turn on agitation and add slowly the Imidocarb dipropionate.

4. Maintain under constant agitation for 30 minutes or until completely dissolved.

5. Add Propionic Acid slowly to this mixture.

6. Maintain under constant agitation for 30 minutes or until completely dissolved.

### MIXTURE B

7. In a previously cleaned and dried container add 10% of the water volume (WFI) 5 L at a temperature of 25-35° C. Maintain the mixture under nitrogen bubbling.

8. Under constant agitation add to the container Sodium Chloride. Maintain under agitation until completely dissolved.

9. Under constant agitation add to the container Cyanocobalamin (Vit-B12). Maintain under agitation until completely dissolved.

10. Under constant agitation add to the container EDTA. Maintain under agitation until completely dissolved.

11. Under constant agitation add to the container Polyethylene glycol 400. Maintain under agitation until completely dissolved. Initials

12. Pour the contents of MIXTURE B into MIXTURE A.

13. Complete the final volume for 100% WFI water (q.s.p.) at a temperature of 25-35° C. Maintain under constant agitation for 60 minutes or until completely homogenized.

14. Collect a sample and verify the pH, which must be between 4.5 and 5.0, and correct it if necessary with Propionic Acid or 5N NaOH Solution.

15. Proceed with the in-line sterile filtration using 0.22 micron cartridge filter.

### Example 2

The following is an example of a formulation consistent with the inventions disclosed herein.

| INGREDIENT | QUANITY gr/L | BATCH gr |
|---|---|---|
| Imidocarb dipropionate | 165.0 | 247.5 |
| Polyethylene glycol 400 | 50.0 | 75.0 |
| Propionic acid | 40.0 | 60.0 |
| Cyanocobalamin vit. B12 | 0.50 | 0.8250 |
| Lidocaine hydrochloride | 12.50 | 18.75 |
| EDTA | 0.1000 | 0.1500 |
| Sodium chloride pa | 8.00 | 12.00 |
| Water for injectables qsp | 1.000 L | 1.500 |

The ingredients were formulated as follows:

### MIXTURE A

1. Check the components of the formula.

2. Collect 50% of the volume of Purified water in the mixture reactor. Start injection of N2 into the liquid.

3. Add polyethylene glycol slowly until completely mixed.

4. Add the propionic acid slowly until completely mixed.

### MIXTURE B

5. Collect 30% of the volume of purified water in another reactor. Start injection of N2 into the liquid.

6. Add the sodium chloride and agitate until completely dissolved.

7. Add the vitamin B12 and continue agitation until completely dissolved.

8. Add Mixture B into A—Complete total volume with water. Agitate until completely homogenized-minimum of 30 minutes.

9. Correct the pH if necessary to 4.5-5.0; always keep the solution in N2 atmosphere.

10. Filter in sterile cartridge 0.22u.

11. Pour into a 20 ml amber bottle and attach the red rubber stopper on—sterile bottles and stoppers.

The above formulation was subjected to accelerated stability testing. 20 ml samples were stored at 42° C. in 75% humidity and tested over a 6 month period by HPLC for Imidocarb Dipropionate content and Vitamin B12 content. The results of the testing was that in all tests, lmidocarb Dipropionate content and Vitamin B12 remained viable with less than a 5% reduction in concentration.

### Example 3

A study was performed with an objective to determine the safety of the formulations as provided in this application after administration in bovines by injection. For this study, the indicated dose was 3.0 mg/kg.

Tested Formulation:
Imidocarb Dipropionate—15 g
Cyanocobalamin (Vit. B12)—0.05 g
Vehicle—100 mL
Route of Administration—Subcutaneous
Dose: 1 ml/50 kg p.v. (3.0 mg/kg of Imidocarb Dipropionate)

For purposes of innocuity and drug tolerance study, every adverse event was noted when identified. The animals were observed during a period of 24 h after the product administration for possible adverse effects. These evaluations were repeated on a daily basis throughout the study period. For this study, adverse effect was considered as any abnormal reaction in the administration site (pain, edema, ulceration), behavior change, loss of appetite and other changes that could be related to the product use.

The study was conducted in bovines for which the drug is recommended to treat hemoparasites. From a lot of 16 animals, 6 crossbred holstein-zebu male bovines without history of treatment with the drugs contained in the tested formulation were selected randomly. The weight of the animals ranged from 165 to 218 Kg. The animals were weighted and identified through numbered ear tags before treatment.

In three prestablished periods, days zero (before treatment), 7 and 15 days post-treatment, the animal blood was collected for analysis of biochemical and hematological parameters searching for hepatic and renal toxicity evidences. The following tests were performed:
Biochemical tests: Urea, Creatinine, Total protein, Alkaline Phosphatase, AST, Gamma Glutamil Transferase (Gamma GT), Creatine kinase, Cholesterol, Magnesium, Calcium, Sodium, Potassium, Chloride;
Hematological tests: Erythrocyte Count, Leukocyte Count, Cellular volume, Mean corpuscular volume (MCV), Mean corpuscular hemoglobin (MCH), Mean corpuscular hemoglobin concentration (MCHC), Differential count of white blood cells, platelets.

Throughout the test, the animals were kept under a semi-confined regimen receiving food and water ad libitum. The food provided, supplementary to pasture, consisted on corn/hay ensilage, concentrate and mineral salt. A maintenance diet was made available for the animals.

The weight of the animals and the administered volume are represented in Table I. All animals were examined through a detailed clinical test.

TABLE 1
Animal Weight and Administered Volume

| | | Formulation |
|---|---|---|
| N | Weight (kg) | Volume (ml) |
| 1 | 192.0 | 3.8 |
| 2 | 165.0 | 3.3 |
| 3 | 180.0 | 3.6 |
| 4 | 198.0 | 4.0 |
| 5 | 203.0 | 4.1 |
| 6 | 218.0 | 4.4 |

Results:

Animals receiving the drug by injection did not show local or systemic reaction signs after administration. The bovines were observed for application site reaction; there was no pain perception in the moment of application or when the administration site was rubbed in all moments when the animals were evaluated after administration. The animals did not present any behavior change, presenting normal clinic behavior and did continue eating and drinking water normally. The weight evolution of the animals remained within the standards expected for animals fed with a maintenance diet. There was no significant change in the analyzed parameters, which supports the behavior clinical evaluation of the test animals and also with the clinical study observations with a higher number of animals during the efficacy and residue studies.

The project had as an objective to assess the innocuity and the waiting period of the formulation containing imidocarb dipropionate, recommended to be administered subcutaneously in bovines.

Results showed that there was no administration site reaction showing any toxic local reaction. Similarly, no clinical behavior change was observed in animals, with no change in the access to food and water, as well as mobility and stimulus reaction.

Results of the biochemical and hematological marker studies confirm the safety of using the product.

We claim:

1. A composition for the treatment of parasitic infectious diseases in livestock comprising imidocarb dipropionate, cyanocobalamin vitamin B-12 and a pharmaceutically acceptable carrier.

2. A method of formulating a composition for the treatment of parasitic infectious diseases in livestock, said formulation comprising imidocarb dipropionate, cyanocobalamin vitamin B-12 and a pharmaceutically acceptable carrier.

3. A method of treating or preventing parasitic infectious diseases in livestock by administering to said livestock a formulation comprising imidocarb dipropionate, cyanocobalamin vitamin B-12 and a pharmaceutically acceptable carrier.
